(19) United States
(12) Patent Application Publication
(10) Pub. No.: US 2012/0316606 A1
(43) Pub. Date: Dec. 13, 2012

(54) DEVICE FOR ENHANCED MECHANICAL STABILIZATION

(76) Inventor: Azadeh Farin, Rolling Hills, CA (US)

(21) Appl. No.: 13/579,252

(22) PCT Filed: Feb. 16, 2011
(86) PCT No.: PCT/US11/25130

§ 371 Date: Aug. 15, 2012

Related U.S. Application Data
(60) Provisional application No. 61/304,951, filed on Feb. 16, 2010.

(51) Int. Cl. A61B 17/86; F16B 35/00

(52) U.S. Cl. 606/305; 411/378

(57) ABSTRACT

The invention relates to a device that provides enhanced mechanical stabilization, as well as methods for its use. In various embodiments, the device is configured in a manner similar to a conventional screw, but with mechanical elements that afford greater fixation and stability and that lessen the likelihood that the device loosens after insertion into a substrate and/or falls out of the substrate entirely or releases its interaction with the substrate. Moreover, the device may have better pullout strength as compared with conventional screws and screw-type devices used in a range of applications.

### BACKGROUND OF THE INVENTION

1. Field of the Invention

The present invention is directed generally to a device that provides enhanced mechanical fixation and stabilization, as well as methods for its use.

2. Description of the Related Art

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Common screws are used for a variety of purposes and in a variety of settings. They range widely in size, in the materials used in their construction, in the configuration of their heads (i.e., to accommodate different screwdrivers and other devices used to actuate them), and in the purposes for which they are employed. Various devices and techniques have been developed to enhance the connection between screws and the substrate into which they are screwed. For instance, adhesives or cements may be added between the screw and the substrate or mechanical devices (e.g., brackets, etc.) may be employed to afford a better grip between a screw and its substrate. Nonetheless, there remains a need in the art for superior and alternative devices, configurations, and methods with more versatility and applicability for enhancing the connection between screws and their substrates, and thus, for providing enhanced mechanical fixation and stabilization among a screw, its substrate and other mechanical elements in communication with either (e.g., an item screwed to a wall, two beams screwed together, or repair of osseous fractures in the human body).

One application of the use of screws is in spinal surgery. Currently, posterior cervical spinal fusion is commonly performed with lateral mass screws at cervical vertebrae C3-C7 and pedicle/translaminar/pars screws at cervical vertebra C2. Thoracolumbar fixation is performed with pedicle screws.

These contemporary devices suffer from a variety of shortcomings, and would therefore benefit from a mechanism that affords greater strength of fixation and stability to withstand pull-out. This enhanced fixation mechanism would reduce the likelihood that the devices loosen after insertion into bone or fall out of bone entirely, resulting in a failed construct. A failed device that is poorly attached to bone could compromise anatomic alignment, result in spinal biomechanical instability, impair neurologic function, and require additional more invasive revision surgery for attempted correction of a potentially dangerous situation. This enhanced feature of improved pull-out strength would improve maintenance and preservation of anatomic alignment and help preserve neurologic function, which is otherwise potentially compromised by such initial factors as spine trauma or spinal deformity.

### SUMMARY OF THE INVENTION

In an embodiment, the invention includes an apparatus, comprising a shaft with exterior threading; a head affixed to the shaft; at least one stabilization apparatus configured in the shaft; and an actuator movably positioned at least partially within the shaft and having an end positioned in the head, the actuator to actuate the at least one stabilization apparatus. The head may further comprise a receiving portion to receive a tool. The head may further comprise a receiving portion adapted to interact with additional apparatuses, rods or other mechanical components. The apparatus may comprise two, three, four or five stabilization apparatuses. The apparatus may comprise at least two stabilization apparatuses that are the same as one another or are each different from one another. Each of the at least one stabilization apparatus may comprise one or more stabilizing elements, wherein upon actuation of each of the at least one stabilization apparatuses, the one or more stabilizing elements each deploy in a generally outward direction from a central axis of the shaft. Each of the at least one stabilization apparatus may comprise at least two stabilizing elements. Each of the at least one stabilization apparatus may comprise three, four or five stabilizing elements. Upon reverse actuation of each of the at least one stabilization apparatuses, the one or more stabilizing elements may each retract into the shaft. The actuator may further comprise a receiving mechanism to receive a tool. The actuator may be adapted to be actuated by rotation relative to the head and the shaft. The actuator may be adapted to be actuated by depression into the head and the shaft. The actuator may comprise a series of ridges parallel to the central axis of the shaft; the stabilization apparatus may comprise at least one stabilizing element each of which may further comprise fingers in mechanical communication with the series of ridges; and the apparatus may be configured such that upon rotation or translation of the position of the actuator the at least one stabilizing element each deploy in a generally outward direction from the central axis of the shaft. The actuator may comprise exterior threading; the stabilization apparatus may comprise at least one stabilizing element each of which may further comprise at least one finger in mechanical communication with the exterior threading on the actuator; and the apparatus may be configured such that upon rotation or translation of the position of the actuator the at least one stabilizing element each deploy in a generally outward direction from the central axis of the shaft. The actuator may comprise exterior threading; the stabilization apparatus may comprise at least one stabilizing element each of which may further comprise a spring-loaded mechanism in mechanical communication with the exterior threading on the actuator; and the apparatus may be configured such that upon rotation or translation of the position of the actuator the at least one stabilizing element each deploy in a generally outward direction from the central axis of the shaft. The actuator may comprise exterior threading in which a mechanical element is incorporated, and the stabilization apparatus may comprise at least one stabilizing element each of which is configured to deploy in a generally outward direction from the central axis of the shaft through a corresponding at least one window in the shaft when in mechanical communication with the mechanical element on the actuator. The at least one stabilization apparatus may be radioopaque and/or visible by X-ray or other imaging approach.

In another embodiment, the invention includes a method of affixing the aforementioned apparatus to a substrate, by providing the substrate; providing the apparatus; positioning an end of the shaft distal from the head against the substrate; rotating the apparatus to drive the apparatus into the substrate; and deploying the stabilization apparatus by rotating the actuator relative to the head and the shaft. The substrate may be selected from the group consisting of a biological material, a nonbiological material, plastic, steel, wood, fiberglass and ceramic. The substrate may be bone. The method may further comprise locking the stabilization apparatus to prevent unintended retraction of the stabilizing elements.

### BRIEF DESCRIPTION OF THE SEVERAL VIEWS OF THE DRAWING(S)

Exemplary embodiments are illustrated in referenced figures. It is intended that the embodiments and figures disclosed herein are to be considered illustrative rather than restrictive.

FIG. 1 depicts a perspective view of a device, in accordance with an embodiment of the present invention.

FIG. 2A depicts a side elevation view of the device with a stabilization apparatus in an undeployed state, and FIG. 2B depicts a cross-sectional view of the device along the line with arrows from FIG. 2A (in the direction of the arrows), in accordance with an embodiment of the present invention.

FIG. 3A depicts a side elevation view of the device with the stabilization apparatus in a deployed state and FIG. 3B depicts a side elevation view of the device with two stabilization apparatuses each in a deployed state, in accordance with embodiments of the present invention.

FIG. 4A depicts a perspective view of the device with the stabilization apparatus in a deployed state, and FIG. 4B depicts a magnified view of the deployed stabilization device, in accordance with an embodiment of the present invention.

FIGS. 5A and 5B depict a top-down view of a stabilization apparatus with five stabilization elements in undeployed and deployed states, respectively, and FIGS. 5C and 5D depict a top-down view of a stabilization apparatus with two stabilization elements in undeployed and deployed states, respectively, in accordance with various embodiments of the present invention.

FIG. 6 depicts a perspective view of a device, in accordance with an embodiment of the present invention.

FIG. 7A depicts a side elevation view of the device with a stabilization apparatus in an undeployed state, and FIG. 7B depicts a cross-sectional view of the device along the line with arrows from FIG. 7A (in the direction of the arrows), in accordance with an embodiment of the present invention.

FIG. 8 depicts a perspective view of the device with the stabilization apparatus in a deployed state, in accordance with an embodiment of the present invention.

FIG. 9A depicts a side elevation view of the device with a stabilization apparatus in a deployed state, and FIG. 9B depicts a cross-sectional view of the device along the line with arrows from FIG. 9A (in the direction of the arrows), in accordance with an embodiment of the present invention.

FIGS. 10A and 10B depict a top-down view of the stabilization apparatus in undeployed and deployed states, respectively, in accordance with an embodiment of the present invention.

FIG. 11 depicts a perspective view of a device, in accordance with an embodiment of the present invention.

FIG. 12A depicts a side elevation view of the device with a stabilization apparatus in an undeployed state, and FIG. 12B depicts a cross-sectional view of the device along the line with arrows from FIG. 12A (in the direction of the arrows), in accordance with an embodiment of the present invention.

FIG. 13 depicts a perspective view of the device with the stabilization apparatus in a deployed state, in accordance with an embodiment of the present invention.

FIG. 14A depicts a side elevation view of the device with a stabilization apparatus in a deployed state, and FIG. 14B depicts a cross-sectional view of the device along the line with arrows from FIG. 14A (in the direction of the arrows), in accordance with an embodiment of the present invention.

FIGS. 15A and 15B depict a top-down view of the stabilization apparatus in undeployed and deployed states, respectively, in accordance with an embodiment of the present invention.

FIG. 16 depicts a perspective view of a device, in accordance with an embodiment of the present invention.

FIG. 17A depicts a side elevation view of the device with a stabilization apparatus in an undeployed state, and FIG. 17B depicts a cross-sectional view of the device along the line with arrows from FIG. 17A (in the direction of the arrows), in accordance with an embodiment of the present invention.

FIG. 18 depicts a perspective view of the device with the stabilization apparatus in a deployed state, in accordance with an embodiment of the present invention.

FIG. 19A depicts a side elevation view of the device with a stabilization apparatus in a deployed state, and FIG. 19B depicts a cross-sectional view of the device along the line with arrows from FIG. 19A (in the direction of the arrows), in accordance with an embodiment of the present invention.

FIGS. 20A and 20B depict a top-down view of the stabilization apparatus in undeployed and deployed states, respectively, in accordance with an embodiment of the present invention.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described.

The invention relates to a device that provides enhanced mechanical fixation and/or stabilization, as well as methods for its use. The terms “stabilization” and “stabilizing” are used herein with reference to both the end result achieved through use of the inventive devices and methods, as well as in the naming of certain elements included within the inventive device (e.g., the “stabilization apparatus” or the “stabilizing elements”). It is to be understood that in each instance where the terms “stabilization” or “stabilizing” are used, they are also meant to capture the concept of “fixation,” because the inventive device and methods accomplish this end result, too. Thus, the elements included within the inventive device also aid in achieving this end. In sum, no conclusions should be drawn nor should any inference be made as to stabilization being achieved without fixation or vice versa, simply by virtue of the use or non-use of one or both of these terms in describing the features of the device.

In various embodiments, the device is configured in a manner similar to a conventional screw, but with mechanical elements that afford greater stability, enhanced fixation and augmented contact and connection between screw and substrate to lessen the likelihood that the device loosens after insertion into a substrate and/or falls out of the substrate entirely. Enhanced fixation due to this novel device minimizes the chance that the integration between screw and substrate can be compromised. Moreover, the device is presumed to have better pullout strength—or resistance to being pulled out—as compared with conventional screws and screw-type devices used in a range of applications.

As will be readily appreciated, the device can be used in a wide array of settings and configurations. It may be used in combination with many other mechanical elements. It can be made in any number of sizes, ranging from very small to very large. It can be constructed of a wide range of materials or combinations of materials, depending upon the desired performance, storage and other characteristics that may be relevant in various circumstances as will be readily appreciated by those of skill in the different arts in which the device is contemplated for use. Simply by way of non-limiting example, the device may be used in building construction, and in such applications might be made of metal, be several inches long, and be used to fasten two pieces of wood together, although these are by no means limitations that must be applied when the inventive device is used in building construction. In other examples, the device might be used in the construction of homes, buildings, furniture, mechanical equipment and a range of industrial applications. In various embodiments, the substrate in which the device is use may be a biological material, a nonbiological material, plastic, steel, wood, fiberglass, ceramic or bone.

In yet another series of examples, the device may be configured for use in human and/or veterinary medical procedures. For example, the device may be used to attach two segments of bone or one or more items to bone anywhere in or on the body of a human or animal. Exemplary applications may be in various spine, orthopedic, hand, maxillofacial, plastic and general surgical procedures; for instance, in spinal surgery procedures, such as in attaching various items to the cervical, thoracic, lumbar or sacral spine or the pelvis or bones of the limbs. In but one example, the device may be used for posterior cervical procedures including stabilization, fusion, or deformity correction. In human and/or veterinary medical procedures, the device may be constructed of metal or any other material or combination of materials suitable for implantation into a human or animal. A range of such materials will be readily appreciated by those of skill in the art. Moreover, because of its design, the device is believed to be biomechanically superior to screws currently available for integration within bone, especially those currently used for spinal fixation.

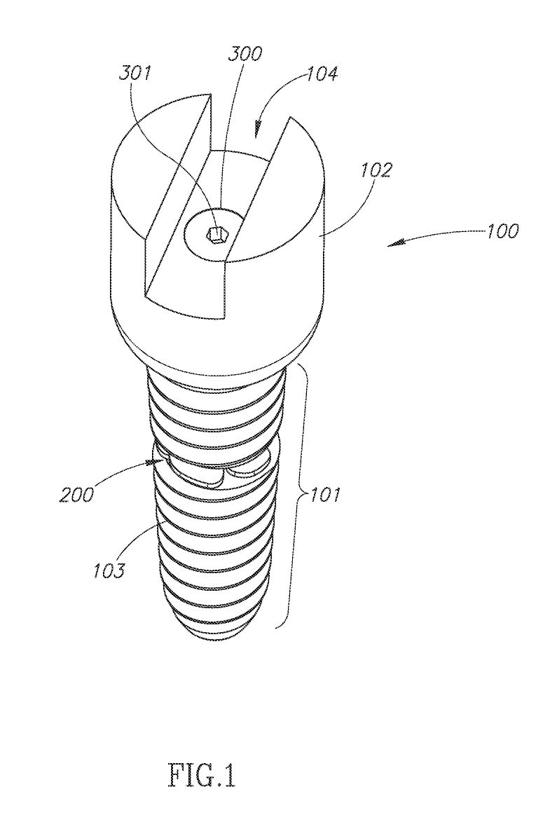

With reference to FIG. 1, in an embodiment of the invention, the device 100 includes a shaft 101 and a head 102. The shaft 101 may include exterior threading 103. The head 102 may include a receiving portion 104 to receive a rotating tool (not shown), whereby rotation of such a rotating tool results in likewise rotation of the device 100. Rotation of the device 100 (with or without axial force being applied, too) causes the device 100 to penetrate and/or burrow into a substrate when in use. In an embodiment, the rotating tool is a screwdriver or similarly-configured device that removably mates with the receiving portion 104 and enables a user of the rotating tool to rotate the device 100, whether by exertion of manual force or via an electromechanical apparatus (e.g., a drill configured with an appropriate bit or an electric screwdriver). In an alternate embodiment (not shown), the rotating tool may be a Phillips-head (or crosshead) screwdriver that removably mates with the receiving portion 104, and in such embodiments the receiving portion 104 may be configured accordingly (i.e., in a x-shaped fashion, akin to the head of a conventional Phillips-head screw). In further, alternate embodiments, the receiving portion 104 may be configured in a variety of manners to accommodate the complementary mechanical elements of the rotating tool(s) that removably mate with the receiving portion 104 and are then used to rotate the device 100. Any number of configurations for the receiving portion 104 are contemplated as being within the scope of the present invention; for instance, the configuration of the receiving portion 104 may be symmetrical or asymmetrical, may be of varying depths, and/or may include multiple depths.

The device 100 is configured to be inserted into a substrate (e.g., bone, wood, metal), in a similar manner as a conventional screw; that is, the exterior threading 103 forces the device 100 into the substrate upon rotation of the device 100 in either the clockwise or counterclockwise direction, depending upon the orientation of the exterior threading 103. The exterior threading 103 may be oriented in any desirable manner; for instance, with a moderate slope in the threading (i.e., with a relatively small angle as measured between the exterior threading 103 and a plane that is perpendicular to the central axis of the shaft 101), or a more severe slope (i.e., with a relatively larger angle as measured between the exterior threading 103 and a plane that is perpendicular to the central axis of the shaft 101).

The head 102 may be of any configuration and/or include any number of mechanical features (not shown) that allow the device 100 to operate for an intended purpose and/or to cooperate with other devices 100 or other apparatuses. Simply by way of example, when used in connection with certain spine surgery procedures, the head 102 may be configured to mechanically interact with a rod that, in turn, can mechanically interact with one or more additional devices 100 or other apparatuses (e.g., conventional screws, etc.). When used in such a fashion, the assembly of one or more devices 100 and one or more rods can connect multiple levels of the spine, similar to the manner in which conventional rod and screw systems that are used for spine surgery operate (e.g., the MOUNTAINEER Occipito-Cervico-Thoracic Spinal System, available from DePuy Companies, or the OASYS System, available from Stryker).

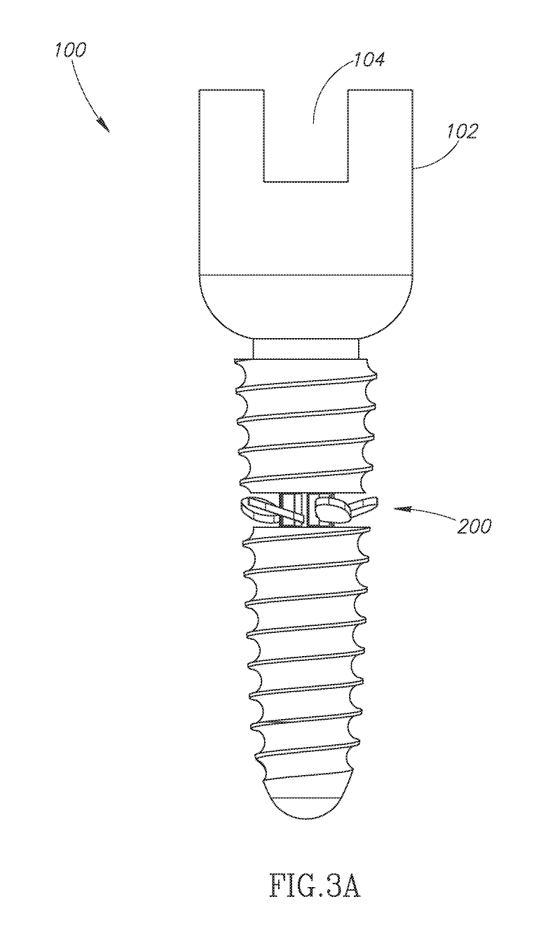

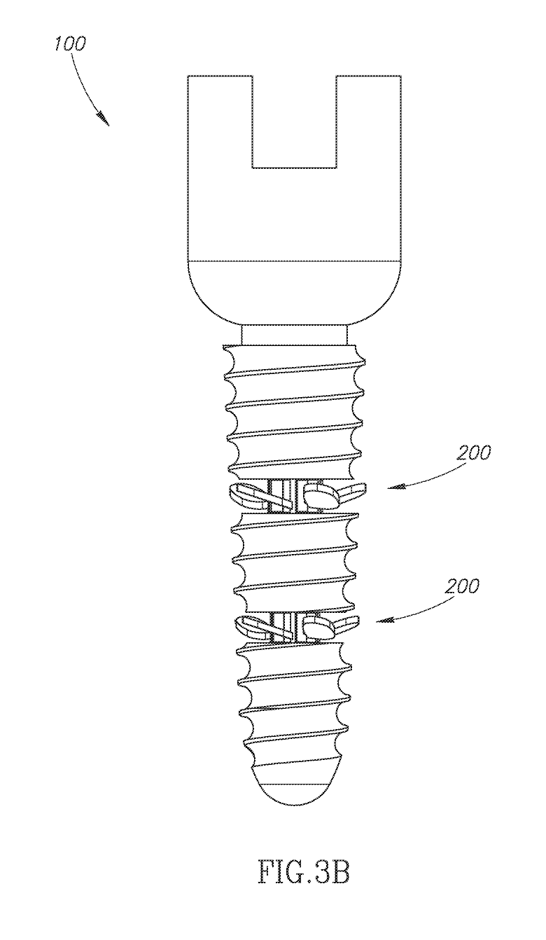

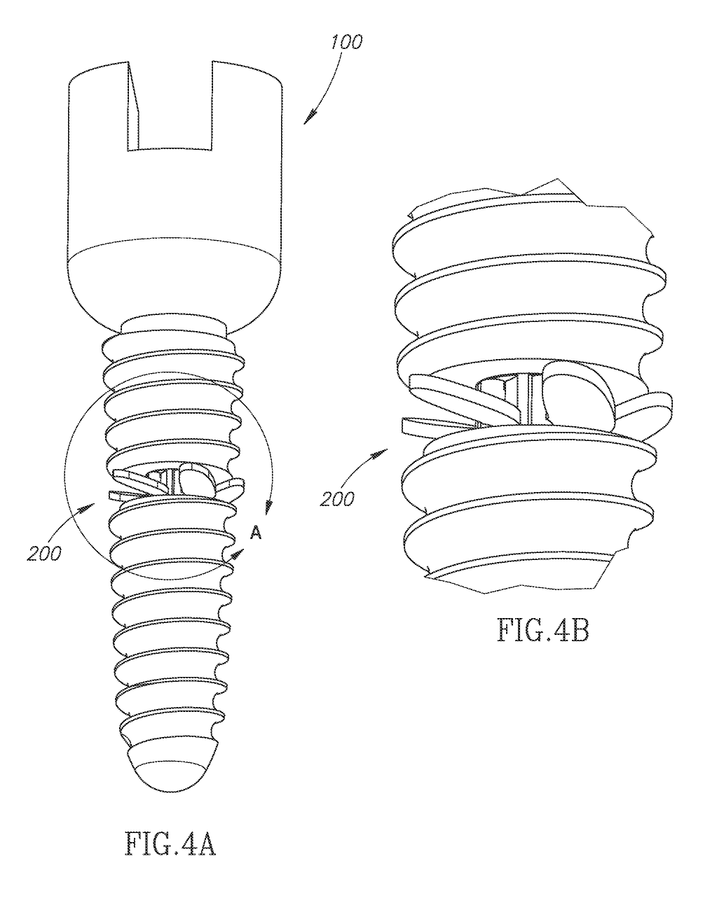

As illustrated in FIGS. 1-4, at least one stabilization apparatus 200 is configured in the shaft 101. In various embodiments of the invention, one, two, three, four, five or more stabilization apparatuses 200 may be configured in the shaft 101; for instance, in FIG. 3B, two stabilization apparatuses 200 are included. In those embodiments where more than one stabilization apparatus 200 is included in the device 100, the stabilization apparatuses 200 may all be the same as one another (as in FIG. 3B), they may all be different from one another, or some may be the same while others are different.

The stabilization apparatus 200 may be configured in a variety of ways. Several examples are illustrated in the drawings, but as will be appreciated by those of skill in the art, there are countless other ways to accomplish the goals of the stabilization apparatus. Among those goals are to augment fixation of the device into a substrate and to provide greater stability to the device; to lessen the likelihood that the device loosens after insertion into a substrate and/or falls out of the substrate entirely; and/or to impart relatively better pullout strength to the device than a conventional screw of similar dimension and material when used in a similar setting. In certain embodiments, the stabilization apparatus does not significantly alter the overall profile of the device in such a manner that it impairs its ability to be inserted into a substrate in the same manner as a conventional screw. For instance, it may be beneficial to minimize disruption to or distortion of the exterior threading.

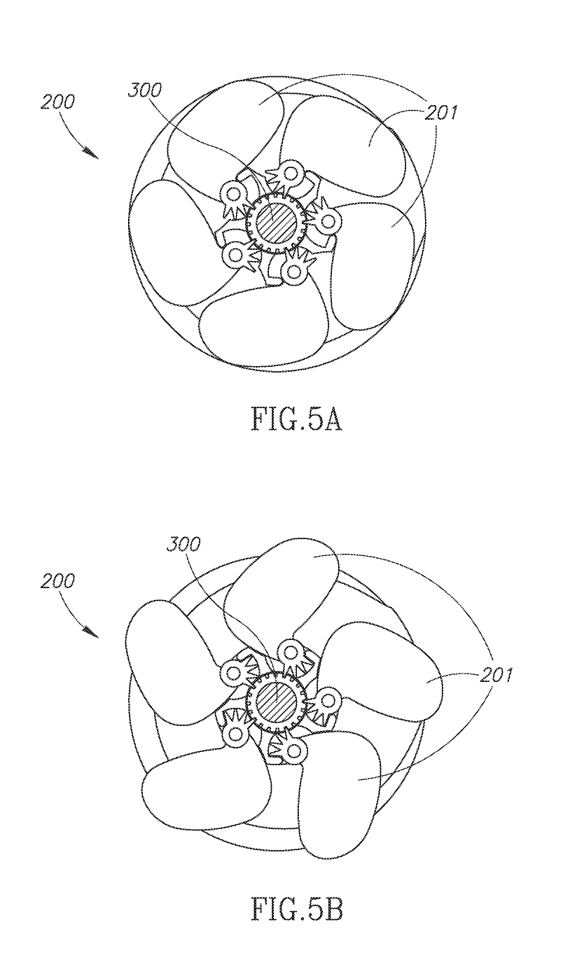

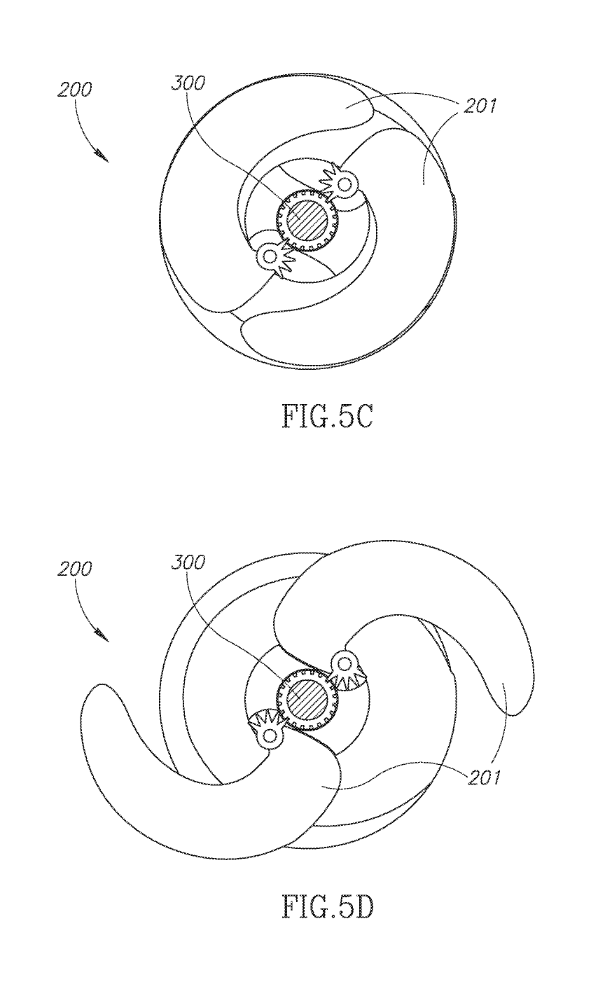

Turning then to the drawings, with reference to FIGS. 1-5, one embodiment of a stabilization apparatus 200 is illustrated. The stabilization apparatus 200 includes a series of stabilizing elements 201 configured to expand in a generally outward direction from the central axis of the shaft 101 upon deployment. The stabilization apparatus 200 depicted in FIGS. 5A and 5B includes five stabilizing elements 201; the stabilization apparatus 200 depicted in FIGS. 5C and 5D includes two stabilizing elements 201; and in yet further embodiments of the invention any number of stabilizing elements 201 may be used, such as one, two, three, four, six, seven, eight or more. In some embodiments, the stabilizing elements 201 are configured not only to be deployed but also retracted back into the shaft 102 (e.g., for removal of the device 100 from a substrate). By way of example, the stabilizing elements 201 depicted in FIG. 5 can all be deployed and retracted back into the shaft 101 by operation (i.e., actuation and reverse actuation, respectively) of the actuator 300, described below.

The stabilizing elements 201 may be of any size, shape, configuration or orientation as may be desirable in a given situation or for a particular application. Simply by way of example, in certain embodiments, it may be desirable to have a greater number of stabilizing elements 201 that extend outward from the shaft 101 a generally short distance, while in other embodiments, it may instead be desirable to have a lesser number of stabilizing elements 201 that extend outward from the shaft 101 a generally longer distance. The stabilizing elements 201 may be blades, spikes, fins or other components that increase the surface area contact between the device 100 and the substrate (e.g., bone, metal, plastic, wood, etc.), thereby increasing resistance to pullout. Those of skill in the art will appreciate a wide variety of configurations for the stabilizing elements 201 of the present invention. All of these embodiments are contemplated as being within the scope thereof.

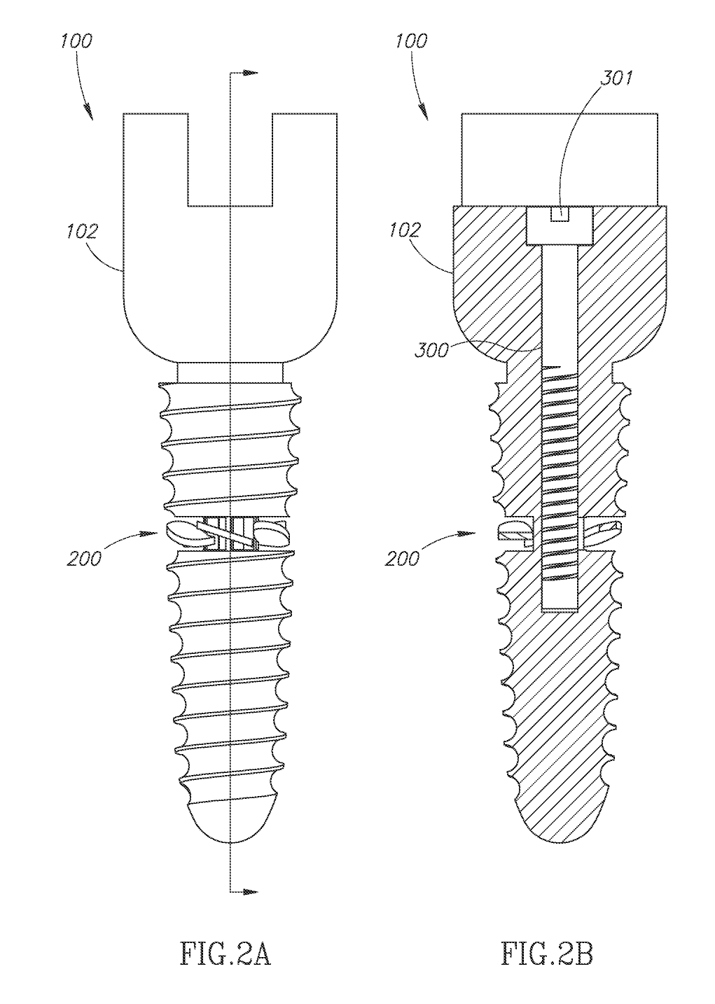

As illustrated in FIGS. 2B and 5, the device 100 may include an actuator 300 configured to actuate the stabilization apparatus 200 and thereby deploy the stabilizing elements 201. In the embodiment illustrated in FIGS. 1-5, the actuator 300 is a rod that extends along at least a portion of the central axis of the shaft 101. The actuator 300 is configured such that rotation or translation or other specific mechanical manipulation of the actuator 300 relative to the head 102 and shaft 101 results in deployment of the stabilizing elements 201. With reference to FIG. 5, this is illustratively accomplished through the interaction of ridges on the actuator 300 with complimentary gears or fingers included in the stabilizing elements 201.

The device may further include a mechanism that enables locking of the stabilization apparatus so as to prevent unintended retraction of the stabilizing elements.

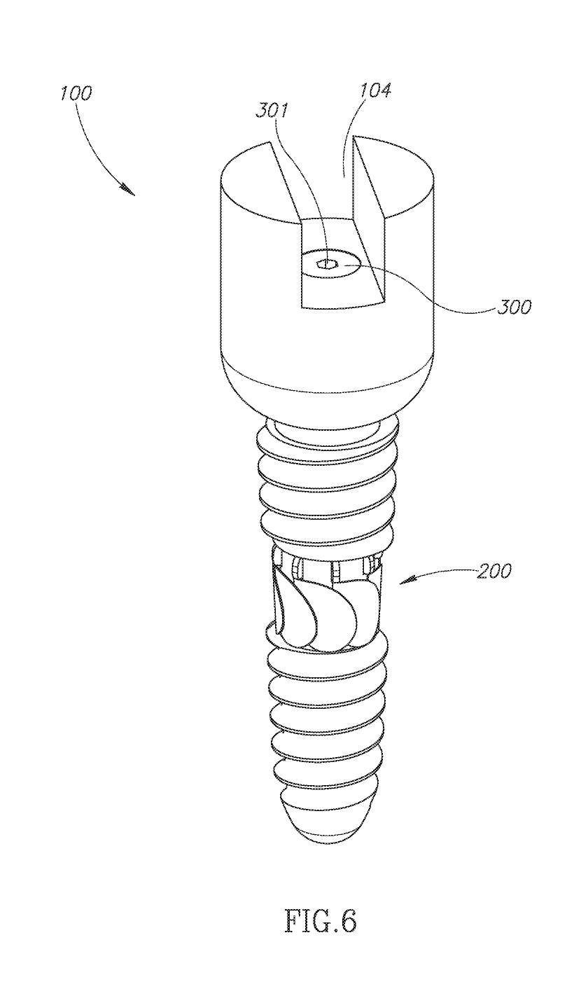

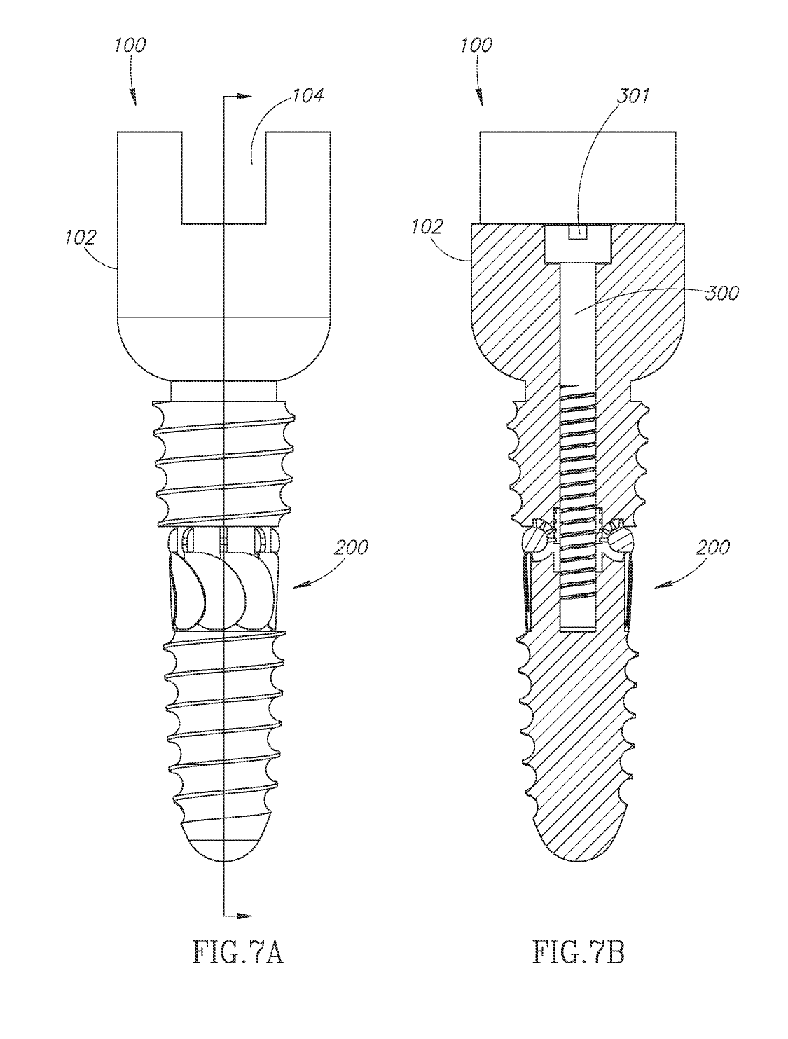

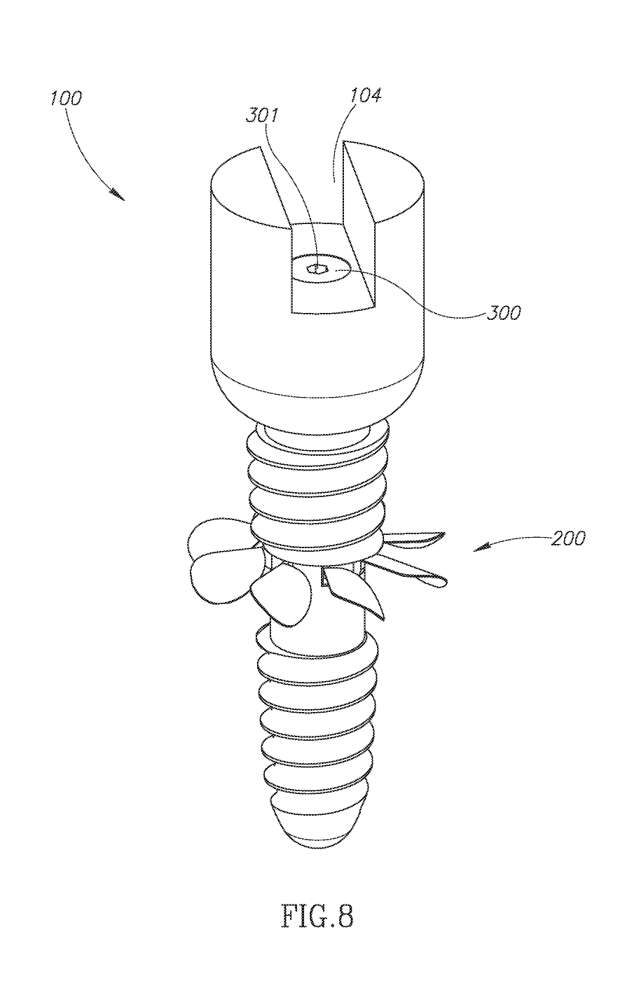

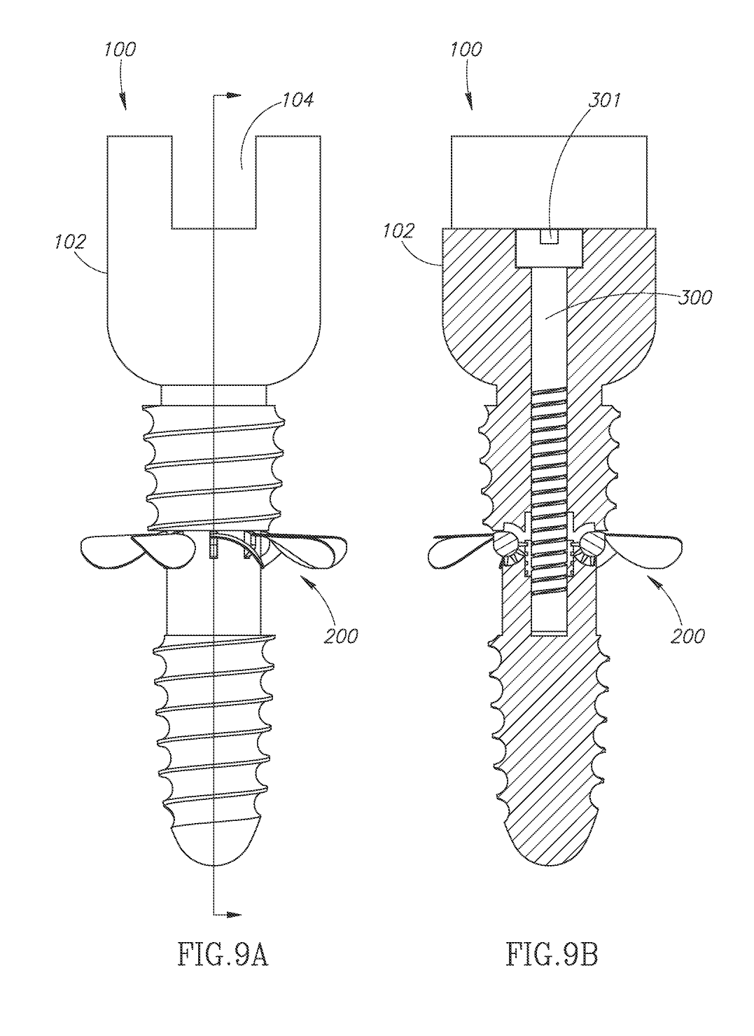

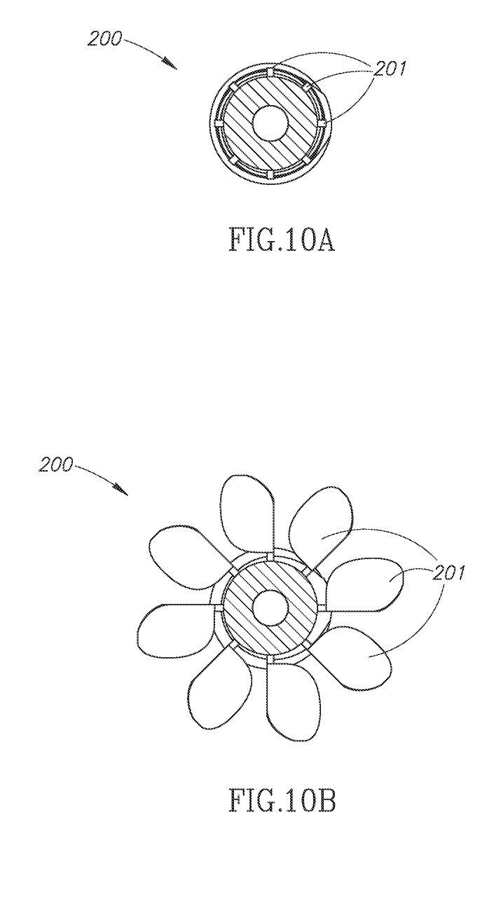

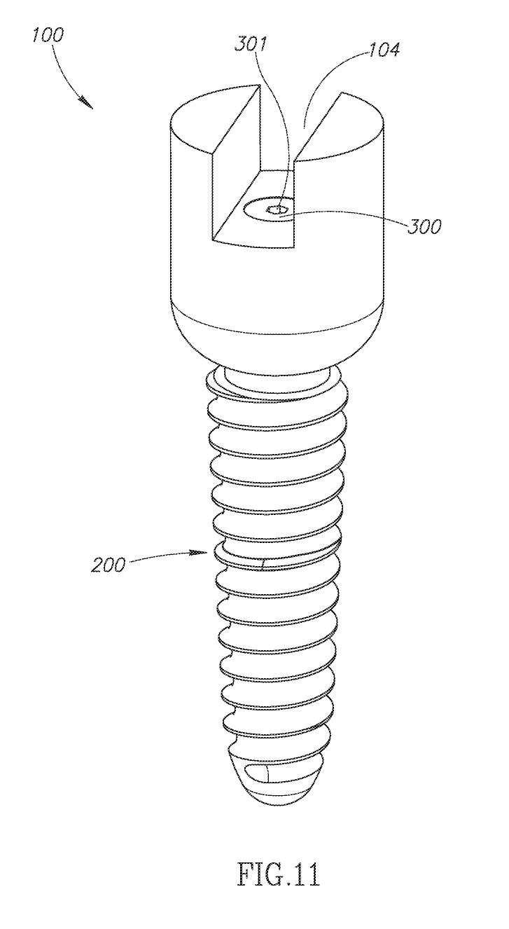

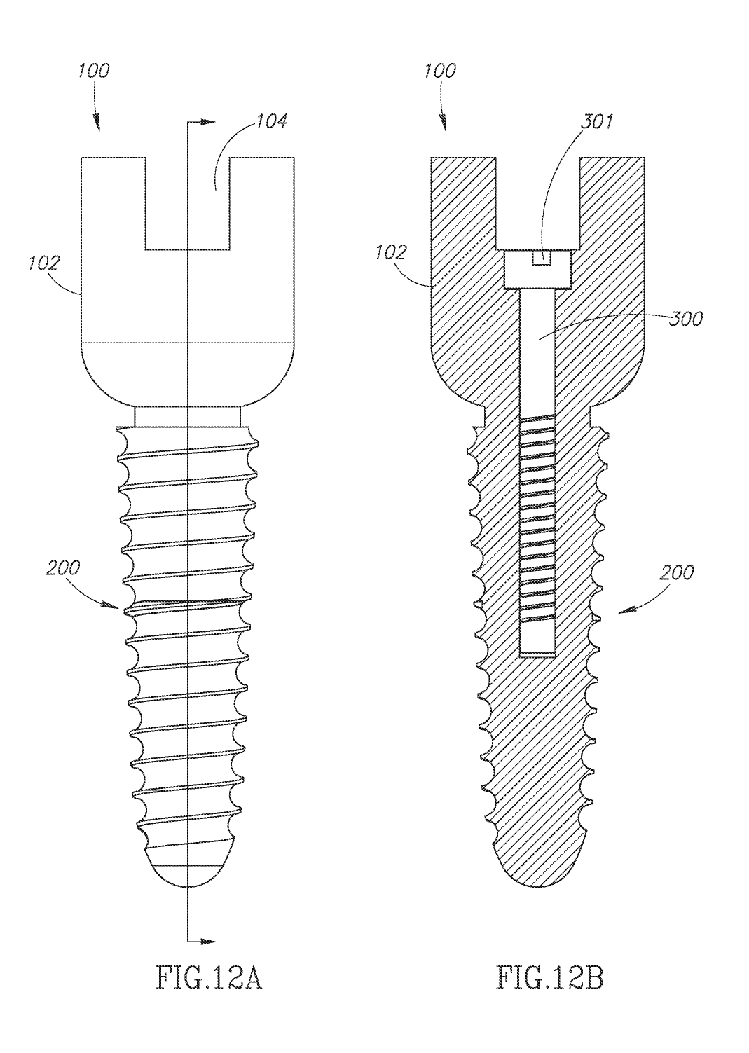

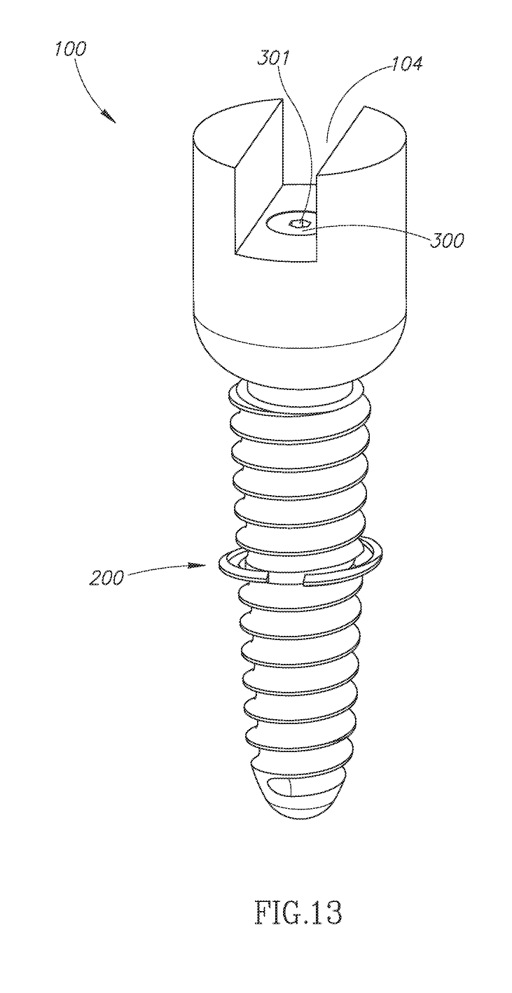

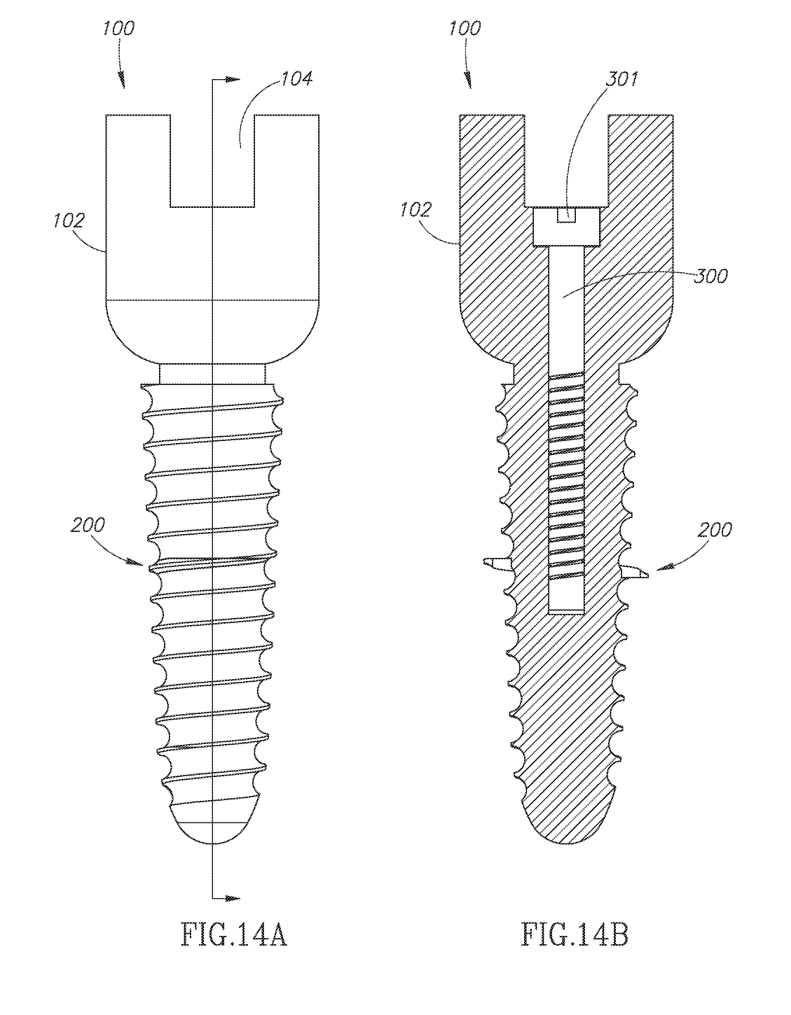

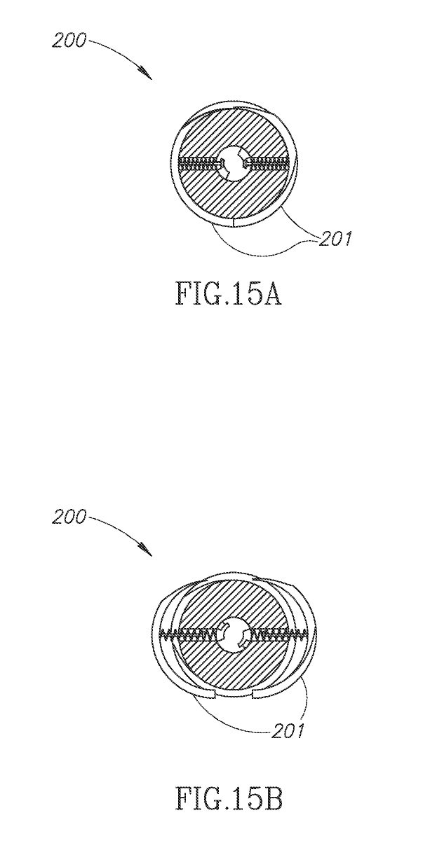

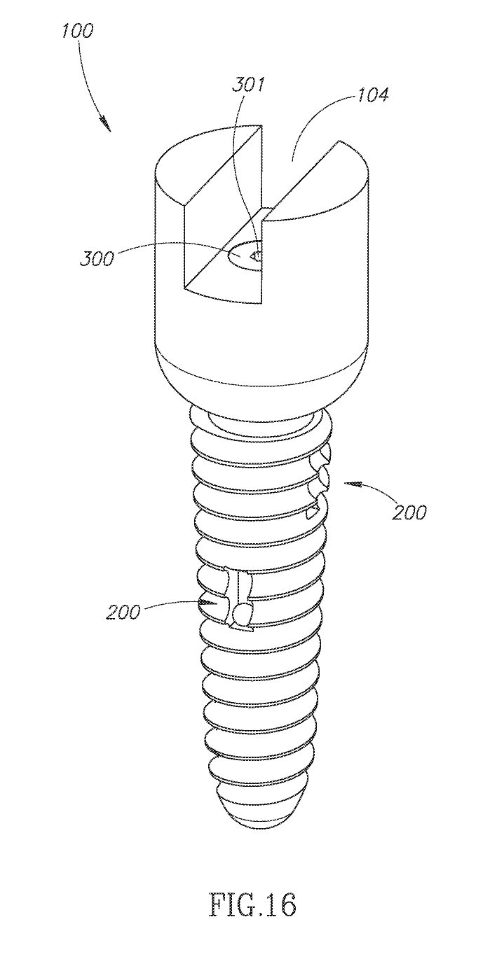

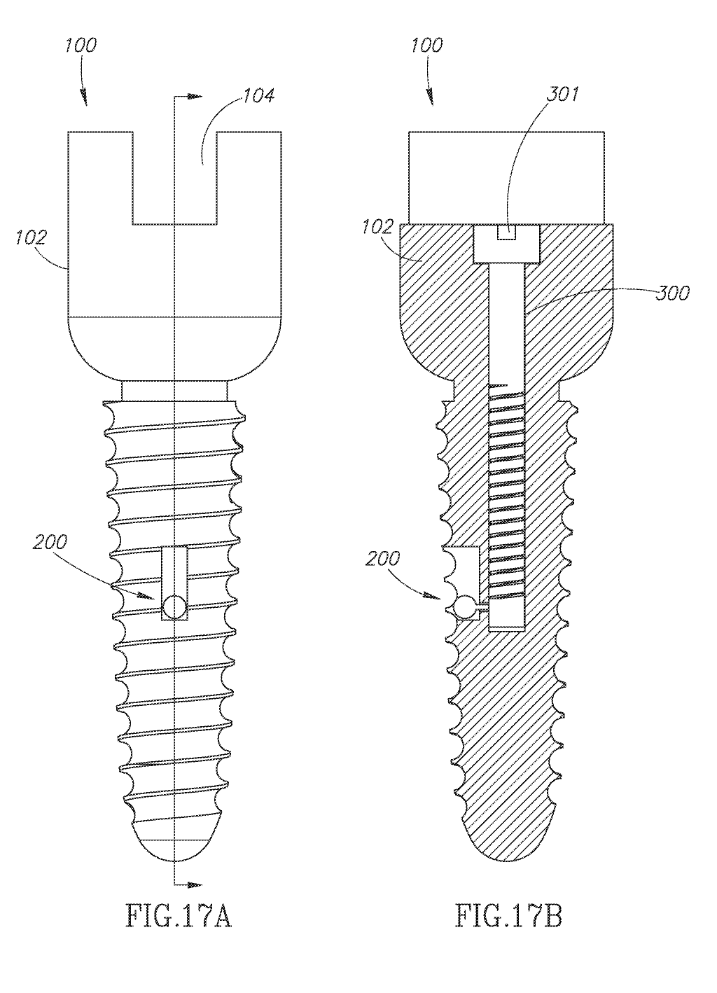

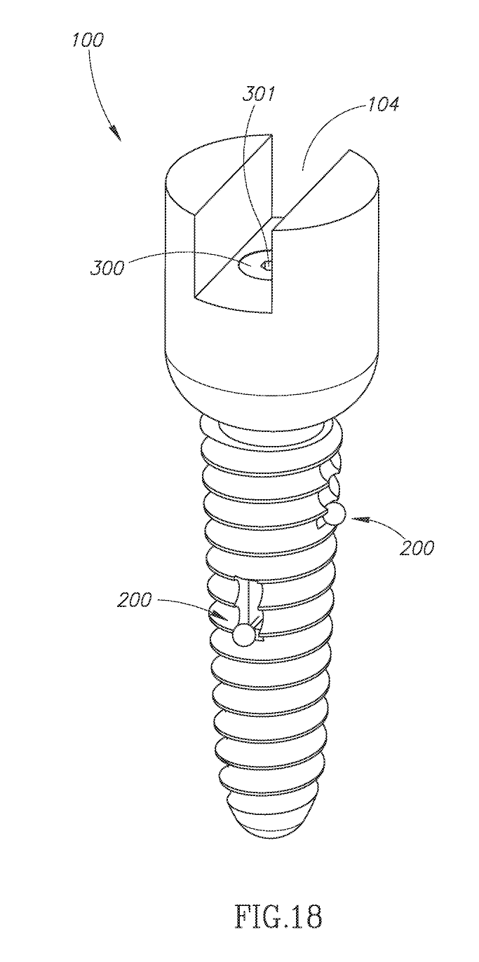

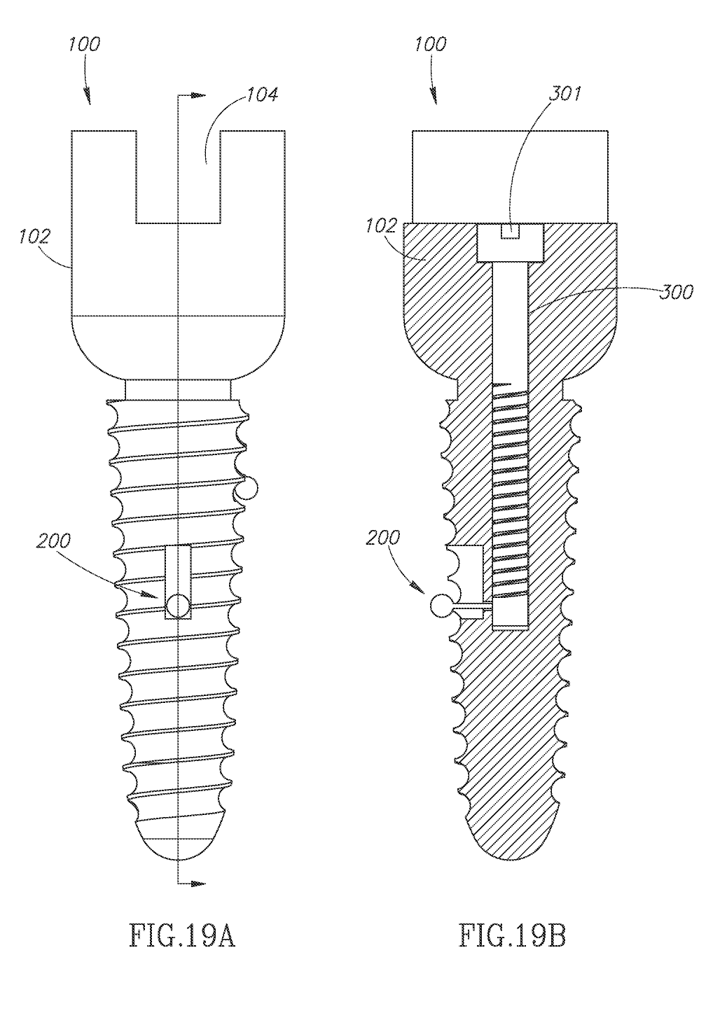

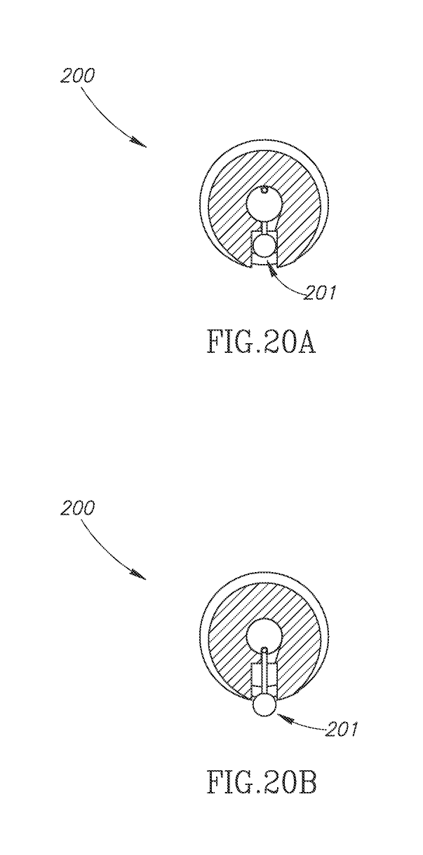

A range of alternate embodiments of the stabilization apparatus and corresponding actuator are illustrated in FIGS. 6-20. For example, in the embodiment illustrated in FIGS. 7B (prior to deployment) and 9B (deployed), threading on the actuator 300 mechanically communicates with fingers included in the stabilizing elements 201, such that, upon rotation or mechanical manipulation of the actuator 300, the stabilizing elements 201 deploy. In another example, in the embodiment illustrated in FIGS. 15A (prior to deployment) and 15B (deployed), a spring-loaded mechanism is released upon rotation or mechanical manipulation of the actuator 300, thereby deploying the stabilizing elements 201 outwardly away from the shaft. In still another example, in the embodiment illustrated in FIGS. 20A (prior to deployment) and 20B (deployed), a mechanical element is incorporated within the screw threading on the actuator 300, such that, when the actuator 300 is rotated or translated such as through minimal downward motion or manipulated otherwise mechanically, then the mechanical element contacts the stabilizing element 201, the stabilizing element 201 is forced through a window in the exterior threading 103 into a deployed configuration.

An actuator tool (not shown) may be used to actuate the actuator 300 through mechanical communication with an actuator receiving mechanism 301. In certain embodiments, the actuator 300 is actuated by rotation relative to the head 102 and/or shaft 101, and discussed above. In alternate embodiments, the actuator 300 may be actuated only by depression of the actuator 300 down into the head 102. Rotation, translation or other mechanical manipulation of the actuator leads to deployment of the stabilizing elements.

A unitary tool may embody both the rotating tool and the actuator tool, such that this one, unitary tool can both mechanically communicate with the receiving portion 104 to enable rotation of the device 100, and also mechanically communicate with the actuator receiving mechanism 301 to deploy the stabilizing elements 201. Simply by way of example, such a unitary tool may be a screwdriver with an additional twist-turn handle, lever, button or other feature with corresponding mechanical elements that controls rotation and/or depression and/or translation or other mechanical manipulation of the actuator 300.

When used in connection with surgical procedures involving the insertion of the device 100 into bone, the deployment of the stabilizing elements 201 may result in a certain amount of friction/resistance to the device 100 pulling out or otherwise loosening or dislodging from the bone depending upon, among other things, how osteoporotic a patient is or how soft the bone/substrate is in relation to the device 100, and how powerfully the pullout force is exerted. Such factors may impact the selection of materials, size and other features of a device 100. Moreover, the stabilizing elements 201 may or may not be totally involved in bone/substrate if the bone is very firm and the blades cannot penetrate it, or they may be simply be in contact with bone/substrate. In other cases, the bone/substrate may be soft in comparison with the stabilization elements 201, and the stabilization elements 201 may thus bite into the bone/substrate. Regardless of the nature of the contact that the stabilization elements 201 make with the bone/substrate, the purpose of the stabilization elements 201 is to increase the pullout strength of the device 100 so that the device 100 is less likely to pull out because the stabilization elements 201 add friction/resistance. Thus, the deployed stabilization elements 201 maintain greater friction against bone/substrate, increase pullout strength, and/or resist pullout. Additionally, the stabilization apparatus 200 and/or stabilizing elements 201 may be radioopaque and/or visible by X-ray or other imaging approach to facilitate insertion, deployment, removal or other procedures as may be desirable and as will be readily appreciated by those of skill in the art.

While not specifically referred to throughout the foregoing discussion, FIGS. 6-10, 11-15 and 16-20 each, respectively, illustrate alternate configurations of the device of the present invention.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Various embodiments of the invention can specifically include or exclude any of these variations or elements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

The invention claimed is:

1. An apparatus, comprising:
a shaft, comprising exterior threading;
a head affixed to the shaft;
at least one stabilization apparatus configured in the shaft; and
an actuator movably positioned at least partially within the shaft and having an end positioned in the head, the actuator to actuate the at least one stabilization apparatus.

2. The apparatus of claim 1, wherein the head further comprises a receiving portion to receive a tool.

3. The apparatus of claim 1, wherein the head further comprises a receiving portion adapted to interact with additional apparatuses, rods or other mechanical components.

4. The apparatus of claim 1, comprising two, three, four or five stabilization apparatuses.

5. The apparatus of claim 1, comprising at least two stabilization apparatuses, and wherein the at least two stabilization apparatuses are the same as one another.

6. The apparatus of claim 1, comprising at least two stabilization apparatuses, and wherein the at least two stabilization apparatuses are each different from one another.

7. The apparatus of claim 1, wherein each of the at least one stabilization apparatus comprises one or more stabilizing elements, and wherein upon actuation of each of the at least one stabilization apparatuses, the one or more stabilizing elements each deploy in a generally outward direction from a central axis of the shaft.

8. The apparatus of claim 7, wherein each of the at least one stabilization apparatus comprises at least two stabilizing elements.

9. The apparatus of claim 8, wherein each of the at least one stabilization apparatus comprises three, four or five stabilizing elements.

10. The apparatus of claim 7, wherein upon reverse actuation of each of the at least one stabilization apparatuses, the one or more stabilizing elements each retract into the shaft.

11. The apparatus of claim 1, wherein the actuator further comprises a receiving mechanism to receive a tool.

12. The apparatus of claim 1, wherein the actuator is adapted to be actuated by rotation relative to the head and the shaft.

13. The apparatus of claim 1, wherein the actuator is adapted to be actuated by depression into the head and the shaft.

14. The apparatus of claim 1, wherein the actuator comprises a series of ridges parallel to the central axis of the shaft; wherein the stabilization apparatus comprises at least one stabilizing element each of which further comprises fingers in mechanical communication with the series of ridges; and wherein the apparatus is configured such that upon rotation or translation of the position of the actuator the at least one stabilizing element each deploy in a generally outward direction from the central axis of the shaft.

15. The apparatus of claim 1, wherein the actuator comprises exterior threading; wherein the stabilization apparatus comprises at least one stabilizing element each of which further comprises at least one finger in mechanical communication with the exterior threading on the actuator; and wherein the apparatus is configured such that upon rotation or translation of the position of the actuator the at least one stabilizing element each deploy in a generally outward direction from the central axis of the shaft.

16. The apparatus of claim 1, wherein the actuator comprises exterior threading; wherein the stabilization apparatus comprises at least one stabilizing element each of which further comprises a spring-loaded mechanism in mechanical communication with the exterior threading on the actuator; and wherein the apparatus is configured such that upon rotation or translation of the position of the actuator the at least one stabilizing element each deploy in a generally outward direction from the central axis of the shaft.

17. The apparatus of claim 1, wherein the actuator comprises exterior threading in which a mechanical element is incorporated; wherein the stabilization apparatus comprises at least one stabilizing element each of which is configured to deploy in a generally outward direction from the central axis of the shaft through a corresponding at least one window in the shaft when in mechanical communication with the mechanical element on the actuator.

18. The apparatus of claim 1, wherein the at least one stabilization apparatus is radioopaque and/or visible by X-ray or other imaging approach.

19. A method of affixing the apparatus of claim 1 to a substrate, comprising the steps of:
providing the substrate;
providing the apparatus;
positioning an end of the shaft distal from the head against the substrate;
rotating the apparatus to drive the apparatus into the substrate; and
deploying the stabilization apparatus by rotating or translating the actuator relative to the head and the shaft.

20. The method of claim 19, wherein the substrate is selected from the group consisting of a biological material, a nonbiological material, plastic, steel, wood, fiberglass and ceramic.

21. The method of claim 19, wherein the substrate is bone.

22. The method of claim 19, further comprising locking the stabilization apparatus to prevent unintended retraction of the stabilizing elements.
